(19) United States
(12) Patent Application Publication
(10) Pub. No.: US 2014/0257100 A1
(43) Pub. Date: Sep. 11, 2014

(54) Porphyrin Microbubbles

(71) Applicant: UNIVERSITY HEALTH NETWORK, Toronto (CA)

(72) Inventors: Gang Zheng, Toronto (CA); Jonathan Lovell, Toronto (CA); Elizabeth Huynh, Mississauga (CA)

(73) Assignee: UNIVERSITY HEALTH NETWORK, Toronto (CA)

(21) Appl. No.: 14/351,354

(22) PCT Filed: Oct. 11, 2012
(86) PCT No.: PCT/CA2012/000937

§ 371 Date: Apr. 11, 2014

Related U.S. Application Data
(60) Provisional application No. 61/546,663, filed on Oct. 13, 2011.

(51) Int. Cl. A61K 47/24; A61B 8/08; A61K 49/22; A61B 5/00; A61K 47/22; A61K 9/14

(52) U.S. Cl. CPC A61K 47/24; A61K 47/22; A61K 9/14; A61K 49/223; A61B 5/0095; A61B 8/481
USPC 600/437; 424/9.52; 424/1.29; 600/407

(57) ABSTRACT

The present invention relates to a microbubble comprising a monolayer of porphyrin-phospholipid conjugate, said microbubble having encapsulated therein, and to the use of said microbubble in ultrasound imaging of a target area in a subject.

### FIELD OF THE INVENTION

This invention relates to the field of microbubbles and, more specifically, to microbubbles formed from porphyrin conjugated to a phospholipid side chain.

### BACKGROUND OF THE INVENTION

Microbubbles are gas-filled microspheres which confer acoustic contrast for ultrasound imaging. Currently, microbubbles are routinely used in the clinic for cardiac diagnosis using echocardiography [1] and are being used for diagnosing cancers and guiding treatment [2], [3]. Ultrasound is one of the most affordable and accessible imaging modalities and therefore any new enabling ultrasound contrast technology holds potential for rapid and widespread impact with existing clinical infrastructure. The expanding field of microbubble research extends beyond diagnostic applications. Preclinical studies are using targeted microbubbles in molecular imaging for assessing pathophysiology [4] as well as in ultrasound-triggered drug and gene delivery [5], [6] for treatment of various diseases and crossing of the blood brain barrier [7], [8].

Clinically used microbubbles are typically formed from fluorinated gases incorporated into a self-assembled phospholipid shell. As such, fluorophores can easily be incorporated into the microbubble shell to verify binding of the microbubble to the target region [9] or for evidence of disruption of the microbubble upon bursting with ultrasound [10]. However, the effect of the presence of these molecules on the stability of the microbubble shell has not been widely investigated and the total fraction of fluorophore relative to the rest of the shell has been minimal. Previously, we introduced a new paradigm for forming a phospholipid bilayer-like structure entirely from porphyrin-conjugated phospholipids [11]. Due to the high porphyrin character, these structures demonstrated unique physical properties, as well as robust intrinsic multimodal imaging properties suitable for fluorescence, photoacoustic, MR and PET.

### SUMMARY OF THE INVENTION

In an aspect, there is provided a microbubble comprising a monolayer with a gas encapsulated therein, the monolayer comprising porphyrin-phospholipid conjugate, wherein the porphyrin-phospholipid conjugate comprises one porphyrin, porphyrin derivative or porphyrin analog covalently attached to a lipid side chain, preferably at the sn-1 or the sn-2 position, of one phospholipid.

In a further aspect, there is provided a method of preparing microbubbles, comprising mixing a gas, a porphyrin-phospholipid conjugate and another phospholipid, wherein the porphyrin-phospholipid conjugate comprises one porphyrin, porphyrin derivative or porphyrin analog covalently attached to a lipid side chain, preferably at the sn-1 or the sn-2 position, of one phospholipid.

In a further aspect, there is provided a method of performing ultrasound imaging on a target area in a subject comprising providing the microbubble described herein; administering the microbubble to the subject; and imaging the target area using ultrasound.

In a further aspect, there is provided a method of imaging a target area in a subject, comprising providing the microbubble described herein; administering the microbubble to the subject; and measuring and/or detecting the photoacoustic signal at the target area. Preferably, the method further comprises bursting the microbubble at the target area prior to measuring and/or detecting the increase in photoacoustic signal.

In a further aspect, there is provided a use of the microbubble described herein for performing imaging.

In a further aspect, there is provided the microbubble described herein for use in performing imaging.

### BRIEF DESCRIPTION OF THE DRAWINGS

Embodiments of the invention may best be understood by referring to the following description and accompanying drawings. In the drawings:

FIG. 1 shows that porphyrin-shell (“porshe”) microbubbles can be formed easily. A) Schematic representation of porphyrin-lipid (top) and porshe microbubbles formed from them encapsulating gas (bottom). B). Image of microbubbles formed with 15 mol % porphyrin-lipid (pyropheophorbide-lipid). C) Straightforward one-pot synthesis to generate porshe microubbles using standard microbubble procedures.

FIG. 2 shows that incorporation of porphyrin-lipid improves yield of microbubble formation using DSPC, PEG40S and a 0.01M PBS buffer. The number of microbubbles was counted as a function of the amount of porphyrin-lipid included in the formulation. Microbubbles were formed from three commonly used fluorinated gases: perfluoropropane (PFP), perfluorobutane (PFB) and sulfur hexafluoride (SF6). Inclusion of 5 to 15% porphyrin-lipid results in optimal microbubble formation for this particular mixture of phospholipids, surfactant and buffer.

FIG. 3 shows that porshe microbubbles have improved stability in serum. Microbubbles were formed with or without 15 molar % porphyrin-lipid and incubated in bovine serum at 37 C. Absorbance represents the number of remaining microbubbles based on light scattering. Porphyrin microbubbles were more stable in serum with a half-life twice as long as microbubbles formed without porphyrin-lipid.

FIG. 4 shows that porphyrin-lipid microbubbles have superior size distribution around 2-3 microns compared with microbubbles formed without porphyrin-lipid. Microbubbles were measured using a Coulter counter. Note the microbubbles formed with porphyrin-lipid have a large and distinct population 2-4 microns in size. Traditional lipid microbubbles often display a polydispersed size distribution.

FIG. 5 shows the acoustic attenuation as a function of frequency for porshe microbub-bles. Grey: 1.5-8.5 MHz transducer. Black: 7-27.5 MHz trans-ducer. Porshe microbubbles exhibit a resonance frequency between 9-10 MHz.

FIG. 6 shows a table of microbubble parameters and shell properties for porshe and commercial lipid microbubbles (literature values). Inclusion of porphyrin-lipid results in an increase in microbubble shell stiffness.

FIG. 7 shows the use of porshe microbubbles for enhanced standard and contrast ultrasound imaging of mouse jugular vein. Porshe microbubbles were injected into a mouse via teil vein injection and both B-mode and contrast mode images were aquired. An 18 MHz transducer frequency was used for imaging.

FIG. 8 shows the use of porshe microbubbles for enhanced contrast ultrasound imaging of a subcutaneous breast cancer (MDA-MB-231) tumor xenograft in a mouse. Porshe microbubbles were injected into a mouse via tail vein injection and both B-mode and contrast mode images were aquired. A 5-12 MHz transducer frequency was used. 1 cm scale bar shown.

FIG. 9 shows the photoacoustic spectrum of porshe microbubbles (mean from 3 measurements +/±SD) acquired from a 5 ns pulsed laser and a 10 MHz single element transducer from a solution of porshe microbubbles.

FIG. 10 shows the photoacoustic and ultrasound properties of porshe microbubbles imaged in a cylindrical well. PA (top) and US (bottom) images of microbubbles (MBs) formed without porphyrin-lipid, porshe MBs and MBs without porphyrin-lipid incubated with equimolar pyropheophorbide-α (pyro) in 9 mm diameter wells within a plastic phantom. PA images were acquired using a 700 nm 5 ns pulsed laser and 10 MHz single element transducer.

### DETAILED DESCRIPTION OF THE PREFERRED EMBODIMENTS

Here we describe “porshe” microbubbles, a new class of microbubbles composed with a porphyrin-phospholipid shell encapsulating a gas, preferably a fluorinated gas.

In an aspect, there is provided a microbubble comprising a monolayer with a gas encapsulated therein, the monolayer comprising porphyrin-phospholipid conjugate, wherein the porphyrin-phospholipid conjugate comprises one porphyrin, porphyrin derivative or porphyrin analog covalently attached to a lipid side chain, preferably at the sn-1 or the sn-2 position, of one phospholipid

Examples of porphyrin-phospholipid conjugates used in forming microbubbles of the application are described in WO 11/044671 by the Applicant.

In an aspect, there is provided a microbubble comprising a monolayer with a gas encapsulated therein, the monolayer comprising porphyrin-phospholipid conjugate, wherein the porphyrin-phospholipid conjugate comprises one porphyrin, porphyrin derivative or porphyrin analog covalently attached to a lipid side chain, preferably at the sn-1 or the sn-2 position, of one phospholipid.

In different embodiments, the porphyrin-phospholipid conjugate may be present at different molar % up to 100 molar %, including 5, 10, 15, 20, 30, 40, 50, 60, 70, 80 and 90 molar %.

In some embodiments, the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophylla, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer. Preferably, the expanded porphyrin is a texaphyrin, a sapphyrin or a hexaphyrin and the porphyrin isomer is a porphycene, an inverted porphyrin, a phthalocyanine, or a naphthalocyanine.

In some embodiments, the phospholipid in the porphyrin-phospholipid conjugate comprises phosphatidylcholine, phosphatidylethanoloamine, phosphatidylserine or phosphatidylinositol. Preferably, the phospholipid comprises an acyl side chain of 12 to 22 carbons.

In some embodiments, the porphyrin in the porphyrin-phospholipid conjugate is pyropheophorbide-a acid.

In some embodiments, the porphyrin in the porphyrin-phospholipid conjugate is a bacteriochlorophyll derivate.

In some embodiments, the phospholipid in the porphyrin-phospholipid conjugate is 1-Palmitoyl-2-Hydroxy-sn-Glycero-3-Phosphocholine or 1-Stearoyl-2-Hydroxy-sn-Gycero-3-Phosphocholine.

In some embodiments, the porphyrin-phospholipid conjugate is pyro-lipid.

In some embodiments, the porphyrin-phospholipid conjugate is oxy-bacteriochlorophyll-lipid.

In some embodiments, the porphyrin is conjugated to the glycerol group on the phospholipid by a carbon chain linker of 0 to 20 carbons.

In some embodiments, the microbubble further comprising a PEGylated emulsifier, preferably having a molecular weight ranging from about 1000 to about 5000. In a preferred embodiment, the PEGylated emulsifier is selected from the group consisting of N-(methoxypolyethylene glycol 5000 carbamoyI)-1,2-dipalmitoyl-sn-glycero-3-phosphatidylethanolamine (MPEG5000-DPPE), 1,2-dimyristoyl-sn-glycero-3-phosphoethanolamine-N-[methoxy(polyethylene glycol)-2000 (DMPE-PEG2000), 1,2-distearoyl-sn-glycero-3-phosphoethanolamine-N-[methoxy(polyethylene glycol)-2000 (DSPE-PEG2000), Polyoxyethylene 40 stearate (PEG40S) and combinations thereof. In certain embodiments, the PEG or PEG-lipid is present in an amount of about 10 molar %.

In some embodiments, the microbubble is substantially spherical and between about 0.8 μm-18 μm in diameter, preferably, between about 1 μm-9 μm in diameter, and further preferably between about 2 μm-4 μm in diameter.

In some embodiments, the porphyrin-phospholipid conjugate comprises a metal chelated therein, optionally a radioisotope of a metal. Preferably, the metal is selected from the group consisting of Zn, Cu, Mn, Fe and Pd.

In some embodiments, the encapsulated gas is a biologically inert gas.

Gases suitable for use in microbubbles include: air, O₂, N₂, H₂, CO₂, N₂O, noble gases, hydrocarbon gases, perfluorocarbon, other fluorinated gases and combinations thereof. Fluorinated gases are preferable because they are not soluble in water which results in greater stability of the microbubble in vitro and in vivo. Other examples of insoluble gases that may be used include sulphur hexafluoride, perfluoromethane, perfluoroethane, perfluoropropane, perfluorobutane and combinations thereof.

Preferred phospholipids used in microbubble formulations contain carbon chain lengths varying from 12-24 carbons and headgroups including phosphatidylcholines, phosphatidylethanolamines, phosphatidic acid and phosphatidylglycerols. In a further embodiment, the phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine(DLgPC), 1,2-dipalmitoyl-sn-glycero-3-[phosphor-rac-(1-glycerol)] (DPPG) and combinations thereof.

The microbubbles can be formed in a solution with a pH buffer and salts of varying type and concentration. Additives may be included in the buffer for increased storage and handling stability. In some embodiments, the microbubble is formed in solution comprising at least one additive selected from the group consisting of Sodium Chloride, Sodium Phosphate, Propylene glycol, Glycerol and Polyethylene glycol (preferably ranging from 1000 to 6000 molecular weight).

In some embodiments, the microbubble further comprises a targeting molecule.

In a further aspect, there is provided a method of preparing microbubbles, comprising mixing a gas, a porphyrin-phospholipid conjugate and another phospholipid, wherein the porphyrin-phospholipid conjugate comprises one porphyrin, porphyrin derivative or porphyrin analog covalently attached to a lipid side chain, preferably at the sn-1 or the sn-2 position, of one phospholipid.

Preferably, prior to mixing with the gas, the porphyrin-phospholipid conjugate is mixed with the other phospholipid to form a lipid film and then hydrated in buffer.

In a further aspect, there is provided a method of performing ultrasound imaging on a target area in a subject comprising providing the microbubble described herein; administering the microbubble to the subject; and imaging the target area using ultrasound.

In a further aspect, there is provided a method of imaging a target area in a subject, comprising providing the microbubble described herein; administering the microbubble to the subject; and measuring and/or detecting the photoacoustic signal at the target area. Preferably, the method further comprises bursting the microbubble at the target area prior to measuring and/or detecting the photoacoustic signal.

In a further aspect, there is provided a use of the microbubble described herein for performing imaging.

In a further aspect, there is provided the microbubble described herein for use in performing imaging.

In some embodiment, the imaging is breast imaging, tumour imaging, carotid neovascularisation imaging, or endoscopic imaging.

The following examples are illustrative of various aspects of the invention, and do not limit the broad aspects of the invention as disclosed herein.

### EXAMPLES

Methods

Formation of Porphyrin-Lipid Microbubbles

Lipid films were prepared in 12 mm×35 mm clear glass threaded vials (Fisher Scientific) by combining 1,2-distearoyl-sn-glycero-3-phosphocholine (DSPC, Avanti Polar Lipids) dissolved in chloroform with porphyrin-lipid (pyropheophorbide-lipid). Films were dried under a stream of nitrogen gas and further dried under vacuum for 30 minutes. Lipid films were stored under argon gas at −20° C. until hydration with 0.01M phosphate buffered saline (150 mM NaCl, 10 mM phosphate, pH 7.4) and polyoxyethylene-40 stearate (PEG40S, Sigma-Aldrich) dissolved in deionized water.

The total lipid concentration was either 0.5 mg/ml or 1 mg/ml in a 0.5 ml volume. The headspace of each vile was filled with gas. Each vile was placed in a 65° C. heated water bath to raise the solution temperature above the transition temperature of the incorporated lipids. The heated vile was sonicated for 10 s (Bransonic Model 2510) to disperse the lipid film into solution and gas was added to the headspace once again. Microbubbles were formed by shaking in a Vial-Mix™ (Bristol-Myers Squibb) for 45 s.

Characterization of Concentration and Size Distribution of Microbubbles

Porphyrin-lipid and regular microbubbles were formulated using porphyrin-lipid at the indicated molar %, 10 molar % PEG40S and the remaining DSPC. The headspace of the vial was filled with sulfur hexafluoride (SF₆, Sigma-Aldrich), perfluorobutane (PFB, Fluoromed L.P) or perfluoropropane (PFP, Fluoromed L.P) gas. After activation of the microbubbles via shaking, microbubbles were used within 6 hours. The size distribution and concentration (number of bubbles/ml) of each microbubble formulation was measured using a Coulter counter system (Multisizer III; Beckman Coulter Inc). Varying volumes of microbubbles were added to 10 ml of Isoton-Il electrolyte solution (Beckman Coulter Inc) to obtain a microbubble count in the range of 100,000-300,000 microbubbles; the dilution was accounted for in the calculation of the microbubble concentration. The number and size distribution were measured using a 30 μm aperture which detected microbubbles with diameters in the range of 0.8 μm to 18 μm. For each microbubble formulation, three samples were measured.

In Vitro Microbubble Stability in Serum

Microbubble stability was compared between microbubbles formed with and without 15 molar % porphyrin-lipid in fetal bovine serum (FBS) at 37° C. Microbubbles were incubated with FBS at a concentration of 4.7×10⁸ microbubbles/ml in a 96-well plate. The absorbance was recorded at 580 nm with a SpectraMax Plus384 plate reader (Molecular Devices) at 0 min, 1 min, 5 min, 10 min, 15 min and then at 15 minute intervals up to 4 hours after the beginning of incubation at 37° C. The plate was mixed for 5 s prior to each measurement. The absorbance is representative of the microbubble concentration. The stability of three samples was measured for each formulation.

Acoustic Characterization of Microbubbles

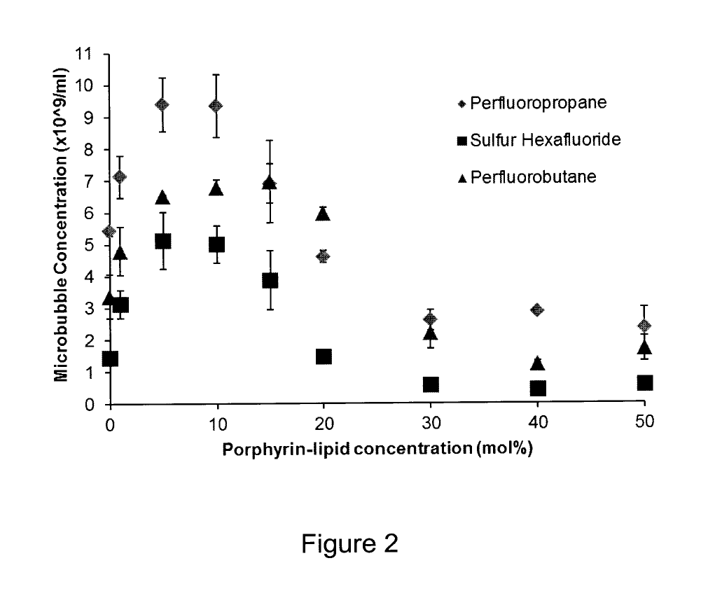

Acoustic characterization was performed with al ml volume of porshe microbubbles composed of 15 molar % porphyrin-lipid, 10 molar % PEG40S, 75 molar % DSPC in a 0.5 mg/ml total lipid concentration with PFP gas. After 1 min decantation, the total volume extracted from the bottom of each vial was 0.5 ml using an 18 gauge needle. Following extraction, the agent was placed within a new 1.5 ml vial, topped with PFP gas and loosely sealed with parafilm (Fisher Scientific). The vial was then gently mixed by hand for 10 s prior to Coulter Counter and attenuation measurements. All measurements were carried out at room temperature. Frequency-dependent attenuation measurements were performed to assess the acoustic response using a method initially described by de Jong et al (de Jong et al. Ultrasonics 1992, 30, 95-103). For this, a narrowband pulse-echo approach was employed, similar to the configuration reported in Goertz et al (Goertz et al. Ultrasound Med Biol 2007, 33, 1376-1388). Two transducers (Model #595396, 5 MHz, 76 mm focus, 12.7 mm diameter, Olympus NDT Canada Inc.; Model #ISO2002HR, 20 MHz, 38 mm focus, 6.35 mm diameter, Valpey-Fisher) were used to cover the frequency range 1.5-27 MHz. The transducers were situated within a water bath and their beams passed through a sample chamber containing diluted agent through a mylar window and were focused upon aluminum reflectors (Supporting FIG. 2). An arbitrary waveform generator (Model AWG5002C, Tektronix) was used to generate pulses with center frequencies spaced at 0.5 MHz intervals. The input waveforms were then amplified by 53 dB (Model A-150, ENI) and then sent to one of the two transducers. On receipt, the echoes were amplified by 35 dB (Model AU1583, Miteq), band-pass filtered and then digitized (400 MHz sampling frequency, Agilent Technologies Inc.) for offline analysis. The peak negative pressure at the focus for all waveforms was calibrated to be 25 kPa using a 0.075 mm diameter needle tip hydrophone (Model 1544, Precision Acoustics). Experiments were conducted with agent diluted (1:7500) in saline (0.9% NaCl) and acoustic measurements commenced at the 1 min following dilution. Experiments were conducted once per sample for a total of 3 samples. Size measurements were performed immediately after having extracted the agent from the vial, and attenuation experiments were performed following the Coulter counter measurements using samples from the same vial extraction. Microbubble shell properties were estimated using the general approach initially described in de Jong et al (N. de Jong, L. Hoff, T. Skotland, N. Bom, Ultrasonics 1992, 30, 95-103.). The specific details of the approach employed here are discussed elsewhere (D. E. Goertz, N. de Jong, A. F. van der Steen, Ultrasound Med Biol 2007, 33, 1376-1388.).

In Vivo Microbubble Ultrasound Imaging of the Mouse Jugular Vein

Animal experiments were conducted in accordance with University Health Network guidelines. Porphyrin-lipid microbubbles were imaged in the jugular vein of female BALB/cJ mice. Hair was removed from the neck of the mouse using a chemical hair remover (Nair; Carter-Horner). The mouse was laid supine on a platform with all legs taped to ECG electrodes for heart rate and respiratory monitoring. The mouse was heated on the plateform for approximately 5 minutes to dilate its blood vessels. A 27G needle was used to inject a 30 μl bolus of microbubbles (˜1.5×10⁹ bubbles/ml) via tail vein. The jugular vein was imaged using the VisualSonics Vevo2100 scanner equipped with an 18 MHz transducer in contrast mode. Images were acquired before the injection of porphyrin-lipid microbubbles and 4s after injection. Image contrast was auto-adjusted linearly.

In Vivo Microbubble Ultrasound Imaging of a Subcutaneous Mouse Tumour

A NIH swiss female athymic nude mouse bearing a subcutaneous MDA-MB-231 tumour in the left flank was injected via the tail vein using a 26 gauge indwelling catheter with a 50 μl bolus of ˜1.5×10⁹ porshe microbubbles/ml followed by a 150 μl saline flush. The mouse was age 12-14 weeks at the time of injection. The tail was warmed using a heated saline pouch to dilate the tail vein prior to catheterization. The tumour was imaged using the Philips iU22 xMATRIX ultrasound system (Philips Medical Systems) and the L12-5 probe with an operating frequency of 5-12 MHz and a mechanical index of 0.07 during simultaneous contrast mode and B-mode images.

Photoacoustic and Ultrasound Imaging of Microbubbles in a Phantom

Photoacoustic characterization was completed using a wavelength-tunable-optical parametric oscillator (OPO) laser (Surelite OPO PLUS; Continuum; wavelength tunability: 650 to 1060 nm) pumped by a Q-switched Nd:YAG laser which was employed to provide laser pulses with a width of 5 ns and a repetition rate of 10 Hz. When the collimated laser pulse traveled through a homemade axicon lens, a ring-shaped light beam was formed. This was focused through an optical condenser and coaxially aligned with the ultrasound focal zone in the water tank. Generated photoacoustic waves were detected by a single-element 10 MHz transducer (V315; Panametrics-NDT), with axial and transverse resolutions of 125 μm and 140 μm, respectively. A sample container was positioned underneath the water tank with an optically and acoustically transparent window covered by a thin transparent membrane coupled with ultrasound gel. Aqueous samples filled the wells that were 9 mm diameter and 3 mm deep. The detected PA signals were first amplified by a low-noise amplifier (5072PR, Panametrics-NDT) and recorded by a digital oscilloscope (TDS 5054, Tektronix). For ultrasound imaging, we utilized the same transducer and raster scanning system as for photoacoustic imaging. In the ultrasound mode, the low-noise amplifier served as both an ultrasound pulse transmitter and receiver. The photoacoustic and ultrasound generation were measured and imaged from three samples: standard microbubbles (0% porphyrin-lipid), porshe microbubbles (15% porphyrin-lipid) and standard microbubbles co-incubated with the same molar concentration of pyro acid as present in 15% porphyrin-lipid microbubbles.

Results

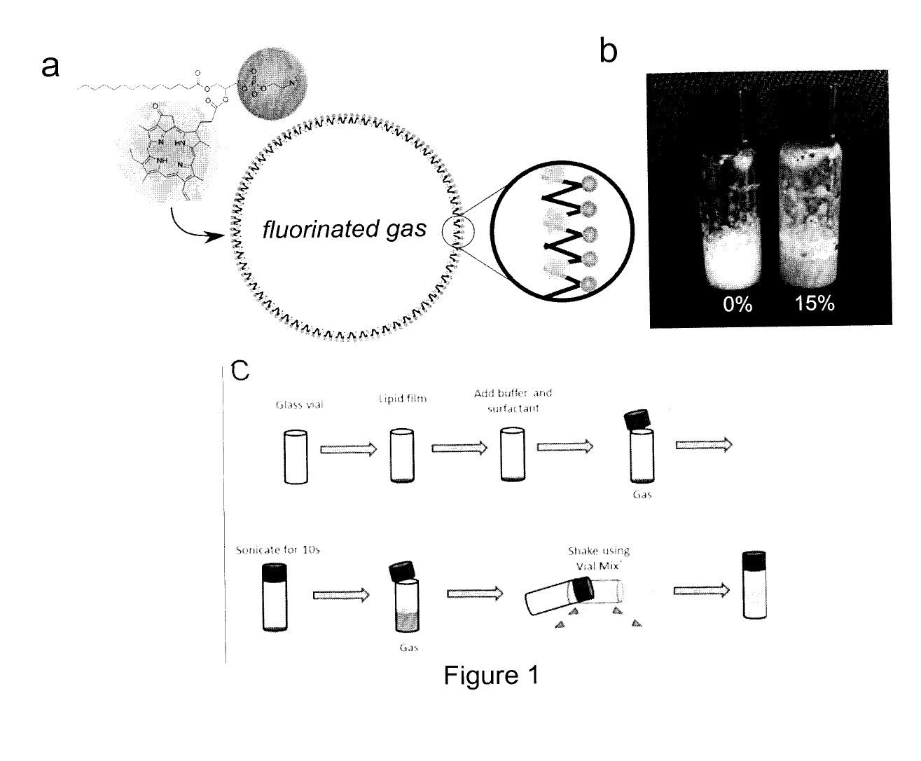

Here we describe a new class of microbubbles composed with a porphyrin-phospholipid shell encapsulating a gas, preferably a fluorinated gas. These porphyrin shell microbubbles “porshe microbubbles” can be easily synthesized using a standard shaking method (FIG. 1) and had a variety of unique physical properties.

When porphyrin-phospholipids were substituted into a standard microbubble formulation at certain concentrations (5-20 molar % porphyrin-phospholipid generated the most bubbles), the resulting microbubbles were found to be formed in higher yield (FIG. 2). Without being bound by any theory, we hypothesize that the porphyrin components stabilize the shell and further prevent gas dissolution, resulting in a greater yield; however, some regular phospholipid was necessary to maintain the high yield. A 15 mol % porphyrin-lipid formulation encapsulating perfluoropropane gas was chosen for further studies, as these MBs were formed in high yield with a relatively large amount of incorporated porphyrin-lipid.

The properties of porshe MBs were compared to MBs formed in PBS without porphyrin-lipid, using only DSPC and PEG40S. To evaluate the influence of the porphyrin-lipid on the MB stability, MBs formed without porphyrin-lipid and porshe

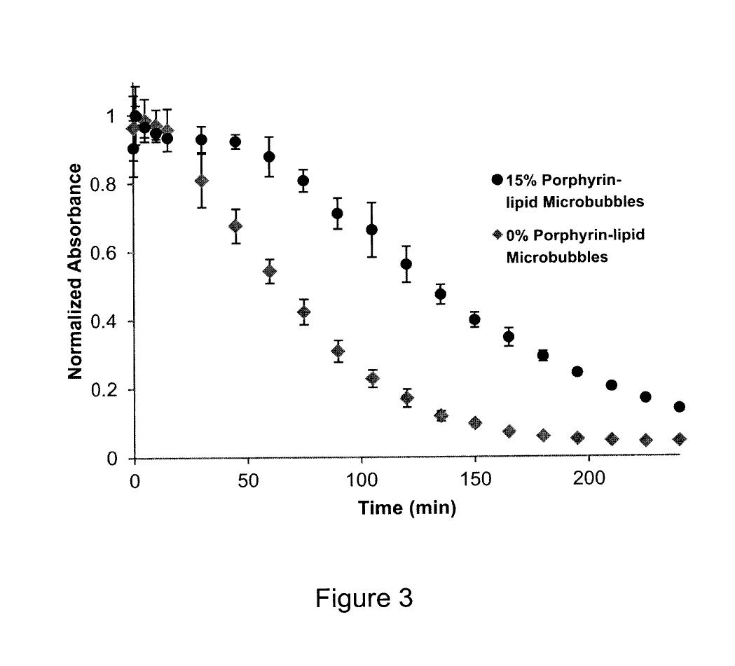

MBs were incubated in fetal bovine serum at 37° C. Optical absorbance due to light scattering was used as a measure of the MB concentration and was monitored over a period of 4 h. Porshe MBs displayed much higher stability than MBs without porphyrin-lipid, remaining nearly twice as long in serum (FIG. 3), further demonstrating that porphyrin-lipid was not only incorporated into the MB shell but also resulted in a more stable MB. Current clinically used microbubbles typically persist for mere minutes in serum in vivo. The longer survival (nearly 2×) of porshe microbubbles, over regular microbubbles in serum, further suggests that porphyrin-lipid stabilizes the microbubble shell. Any lengthening of circulation times is highly desirable.

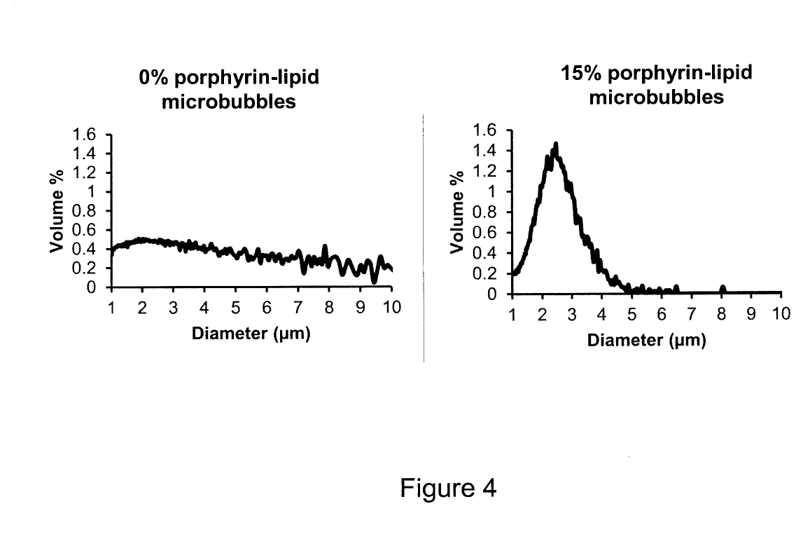

It is recognized that monodispersed microbubbles generate a superior ultrasound signal in comparison to polydispersed microbubble populations [12]. Our porphyrin shell microbubbles were shown to have superior monodispersity (FIG. 4). High monodispersity is a microbubble property that is highly desirable yet difficult to obtain. This level of monodispersity was only observed when porphyrin-lipid was incorporated into the shell. It is notable that this monodispersed size population was achieved with a simple fabrication method with no further purification step.

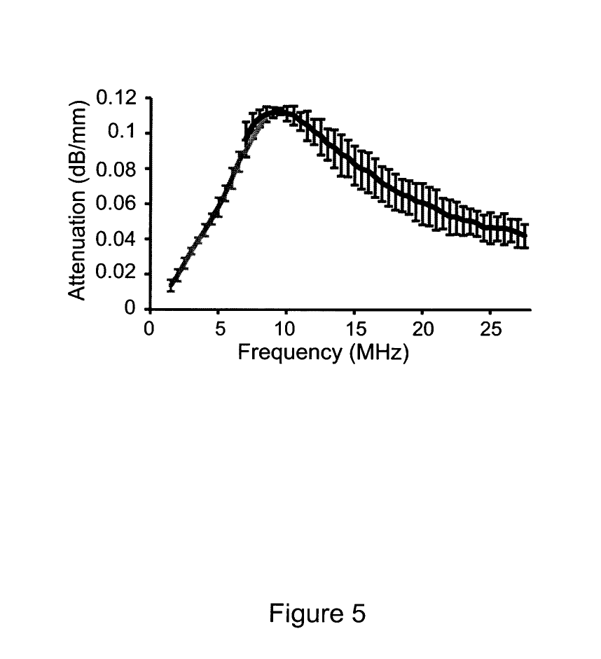

The narrow volumetric size distribution of porshe MBs produced a sharp rise in US attenuation with increasing frequency, which exhibited a peak at 9-10 MHz and gradually tapered off by 27.5 MHz (FIG. 5). having a higher resonance frequency (f res) than some commercial lipid-based MBs, porshe MBs may be well-suited for imaging applications exploiting the 5-15 MHz frequency range, including breast imaging, carotid neovascularization, superficial tumors, and endoscopic imaging.

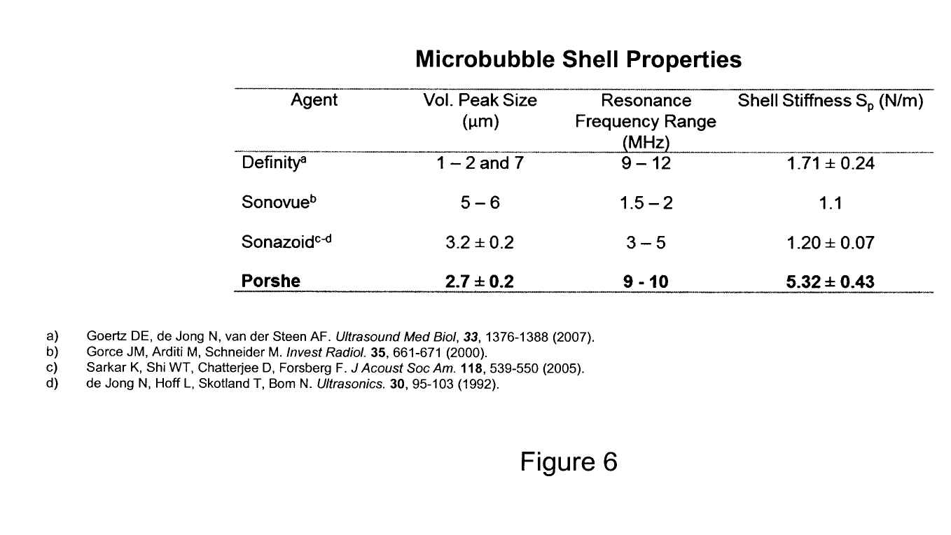

The mechanical properties of porshe microbubbles were measured using an acoustic theoretical model introduced by de Jong et al [13] and input data from the acoustic attenuation (FIG. 5) and the size distribution (FIG. 4). Estimates of the shell stiffness have been previously reported for several commercial lipid MBs, and the porshe MBs were found to be 3-5 times stiffer than these agents (FIG. 6), suggesting that a stiffer shell results in a more stable microbubble as a result of decreased gas permeability and a greater resistance to collapse.

The porphyrin shell microbubbles are also intrinsically suited for multimodal imaging. A large number of porphyrins are integrated into the porphyrin shell (over 1 million porphyrins for a typical 15 mol % microbubble composition). This makes them suitable for photoacoustic imaging methods, hybrid PET or MR imaging approaches, or new ultrasound guided optical imaging techniques.

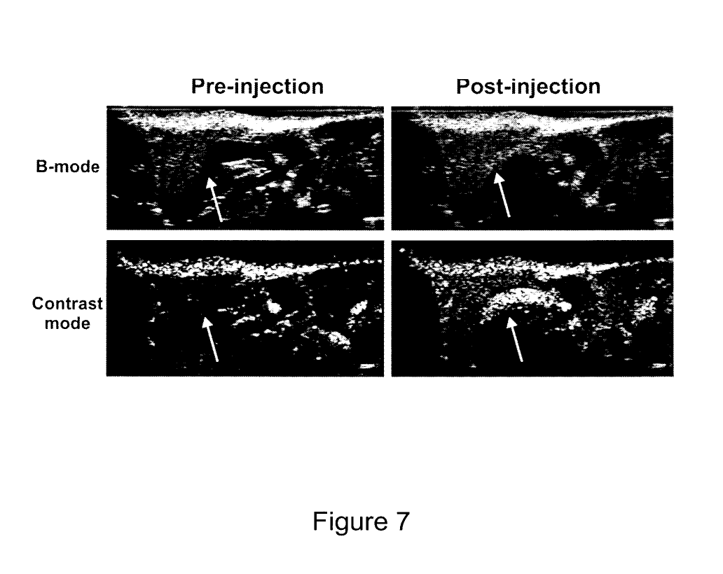

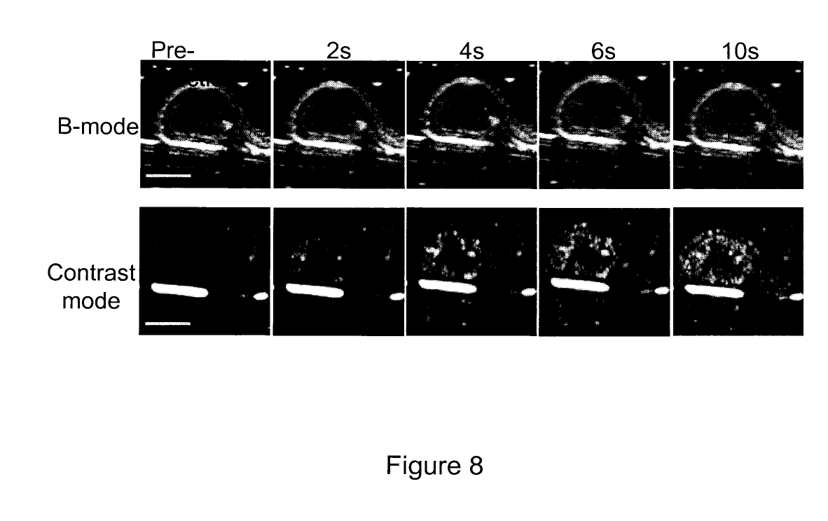

Porshe MBs maintained the nonlinear properties associated with lipid MBs and provided ultrasound contrast. Porshe microbubbles were able to distinguish the jugular vein in a healthy mouse (FIG. 7) and image a subcutaneous tumour in a mouse bearing a human breast cancer (MDA-MB-231) xenograft after intravenous injection (FIG. 8).

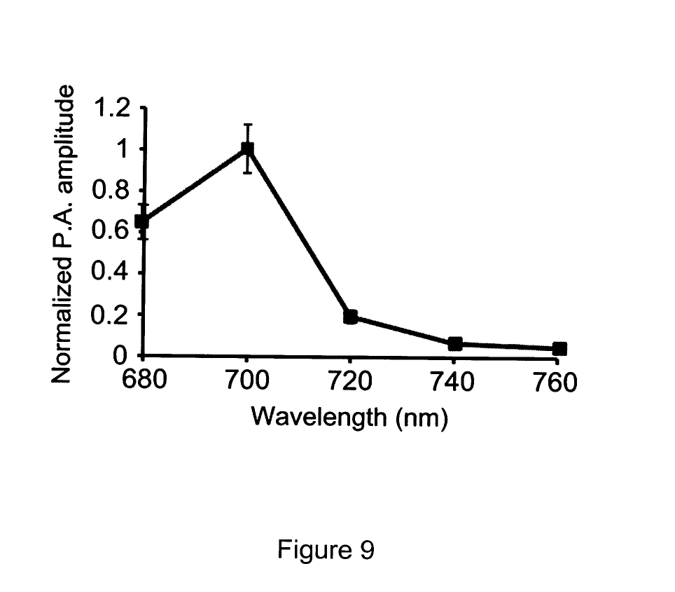

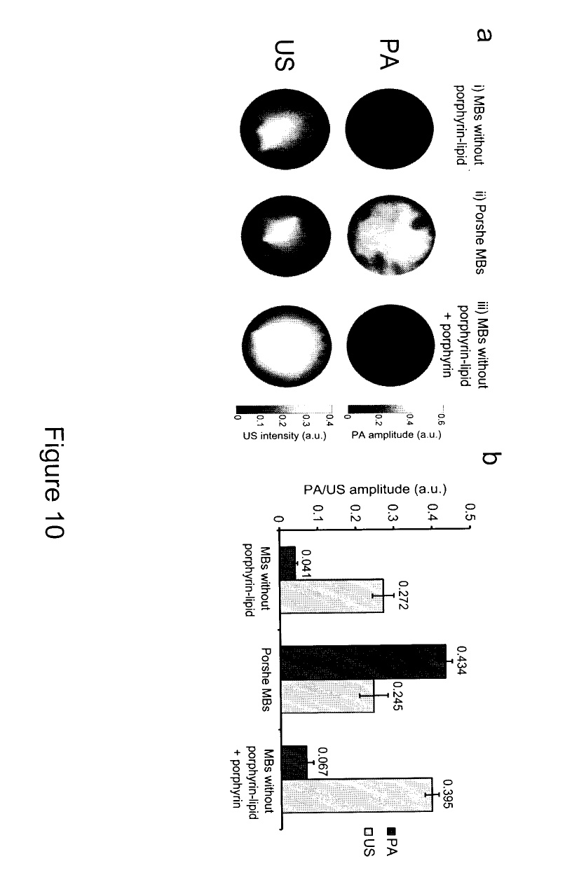

The presence of porphyrin-lipid enables porshe MBs to generate a photoacoustic signal that peaks at 700 nm (FIG. 9). MBs formed without porphyrin-lipid, porshe MBs, and MBs without porphyrin-lipid coincubated with equimolar free porphyrin (pyropheophorbide) were photoacoustically imaged in sample wells using a 700 nm, 5 ns pulsed laser and a 10 MHz single-element US transducer (FIG. 10a top). The porshe MBs generated a 10-fold greater PA signal than MBs formed without porphyrin-lipid and a 6-fold greater PA signal than MBs without porphyrin-lipid mixed with free porphyrin (FIG. 10b). This demonstrates not only that the porphyrin must be present in order for the MBs to generate a PA signal but also that the porphyrin must be conjugated to the lipid and present within the MB shell. All of the MB populations generated strong US signals (FIG. 10a bottom, FIG. 10b), confirming that porshe MBs have the capacity to serve as a dual-modality US/PA contrast agent.

Bursting of the porphyrin-lipid microbubbles can be accomplished using high power ultrasound or sonication. Upon microbubble bursting, small porphyrin-lipid assemblies (nanometer scale) would be formed which may or may not maintain the close packing density of the porphyrin-lipid. Bursting of the porphyrin-lipid microbubbles could be useful in applications relating to medical imaging and drug delivery by externally triggered delivery of the smaller porphyrin-lipid assemblies to soft tissue.

These independent but complementary advantageous traits make porshe microbubbles ideally-suited to become an improved imaging agent for both existing ultrasound imaging, as well as emerging multimodal imaging approaches (e.g. photoacoustic imaging).

Although preferred embodiments of the invention have been described herein, it will be understood by those skilled in the art that variations may be made thereto without departing from the spirit of the invention or the scope of the appended claims. All references mentioned herein, including in the following reference list, are incorporated in their entirety by reference.

### REFERENCES

[1] S. Liu and M. A. Vannan, “Use of Echo Contrast in Routine Practice,” in Case Based Echocardiography, T. Abraham, Ed. London: Springer London, 2010, pp. 117-125.
[2] F. Moriyasu and K. Itoh, “Efficacy of Perflubutane Microbubble-Enhanced Ultrasound in the Characterization and Detection of Focal Liver Lesions: Phase 3 Multicenter Clinical Trial,” American Journal of Roentgenology, vol. 193, no. 1, pp. 86-95, July 2009.
[3] H. P. Weskott, “Emerging roles for contrast-enhanced ultrasound,” Clinical Hemorheology and Microcirculation, vol. 40, no. 1, pp. 51-71, January 2008.
[4] A. L. Klibanov, “Ligand-Carrying Gas-Filled Microbubbles: Ultrasound Contrast Agents for Targeted Molecular Imaging,” Bioconjugate Chemistry, vol. 16, no. 1, pp. 9-17.
[5] A. Lawrie, A. F. Brisken, S. E. Francis, D. C. Cumberland, D. C. Crossman, and C. M. Newman, “Microbubble-enhanced ultrasound for vascular gene delivery,” Gene Therapy, vol. 7, no. 23, pp. 2023-2027, December 2000.
[6] S. Hernot, “Microbubbles in ultrasound-triggered drug and gene delivery,” Advanced Drug Delivery Reviews, vol. 60, no. 10, pp. 1153-1166.
[7] M. Kinoshita, N. McDannold, F. A. Jolesz, K. Hynynen, “Noninvasive localized delivery of Herceptin to the mouse brain by MRI-guided focused ultrasound-induced blood-brain barrier disruption,” Proceedings of the National Academy of Sciences, vol. 103, no. 31, pp. 11719-11723, August 2006.
[8] M. Kinoshita, N. McDannold, F. A. Jolesz, K. Hynynen, “Targeted delivery of antibodies through the blood-brain barrier by MRI-guided focused ultrasound,” Biochemical and Biophysical Research Communications, vol. 340, pp. 1085-1090,
[9] J. R. Lindner, J. Song, J. Christiansen, A. L. Klibanov, F. Xu, and K. Ley, “Ultrasound Assessment of Inflammation and Renal Tissue Injury With Microbubbles Targeted to P-Selectin,” Circulation, vol. 104, no. 17, pp. 2107-2112, October 2001.
[10] D. M. Skyba, R. J. Price, A. Z. Linka, T. C. Skalak, and S. Kaul, “Direct In Vivo Visualization of Intravascular Destruction of Microbubbles by Ultrasound and its Local Effects on Tissue,” Circulation, vol. 98, no. 4, pp. 290-293, July 1998.
[11] J. F. Lovell et al., “Porphysome nanovesicles generated by porphyrin bilayers for use as multimodal biophotonic contrast agents,” Nat Mater, vol. 10, no. 4, pp. 324-332, April 2011.
[12] J. E. Streeter, R. Gessner, I. Miles, and P. A. Dayton, “Improving sensitivity in ultrasound molecular imaging by tailoring contrast agent size distribution: in vivo studies,” Molecular Imaging, vol. 9, no. 2, pp. 87-95, April 2010.
[13] de Jong, N.; Hoff, L.; Skotland, T.; Born, N, “Absorption and scatter of encapsulated gas filled microspheres: theoretical considerations and some measurements,” Ultrasonics, vol. 30, no. 2, pp. 95-103, March 1992.

1. A microbubble comprising a monolayer with a gas encapsulated therein, the monolayer comprising porphyrin-phospholipid conjugate, wherein the porphyrin-phospholipid conjugate comprises one porphyrin, porphyrin derivative or porphyrin analog covalently attached to a lipid side chain, preferably at the sn-1 or the sn-2 position, of one phospholipid.

2. The microbubble of claim 1 comprising up to 100 molar % porphyrin-phospholipid conjugate.

3. The microbubble of claim 1 comprising up to 40 molar % porphyrin-phospholipid conjugate.

4. The microbubble of claim 1 comprising up to 30 molar % porphyrin-phospholipid conjugate.

5. The microbubble of claim 1 comprising up to 20 molar % porphyrin-phospholipid conjugate.

6. The microbubble of claim 1 comprising between 5-20 molar % porphyrin-phospholipid conjugate.

7. The microbubble of claim 1 comprising between 5-15 molar % porphyrin-phospholipid conjugate.

8. The microbubble of claim 1 comprising between 10-15 molar % porphyrin-phospholipid conjugate.

9. The microbubble of claim 1 comprising about 15 molar % porphyrin-phospholipid conjugate.

10. The microbubble of claim 1 wherein the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophylla, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer.

11. The microbubble of claim 10, wherein the expanded porphyrin is a texaphyrin, a sapphyrin or a hexaphyrin and the porphyrin isomer is a porphycene, an inverted porphyrin, a phthalocyanine, or a naphthalocyanine.

12. The microbubble of claim 1 wherein the phospholipid in the porphyrin-phospholipid conjugate comprises phosphatidylcholine, phosphatidylethanoloamine, phosphatidylserine or phosphatidylinositol.

13. The microbubble of claim 12, wherein the phospholipid comprises an acyl side chain of 12 to 22 carbons.

14. The microbubble of claim 1 wherein the porphyrin in the porphyrin-phospholipid conjugate is pyropheophorbide-a acid.

15. The microbubble of claim 1 wherein the porphyrin in the porphyrin-phospholipid conjugate is a bacteriochlorophyll derivate.

16. The microbubble of claim 1 wherein the phospholipid in the porphyrin-phospholipid conjugate is 1-Palmitoyl-2-Hydroxy-sn-Glycero-3-Phosphocholine or 1-Stearoyl-2-Hydroxy-sn-Gycero-3-Phosphocholine.

17. The microbubble of claim 1 wherein the porphyrin-phospholipid conjugate is pyro-lipid.

18. The microbubble of claim 1 wherein the porphyrin-phospholipid conjugate is oxy-bacteriochlorophyll-lipid.

19. The microbubble of claim 1 wherein the porphyrin is conjugated to the glycerol group on the phospholipid by a carbon chain linker of 0 to 20 carbons.

20. The microbubble of claim 1 further comprising a PEGylated emulsifier.

21. The microbubble of claim 19 wherein the PEGylated emulsifier has a molecular weight ranging from about 1000 to about 5000.

22. The microbubble of claim 19, wherein the PEGylated emulsifier is selected from the group consisting of N-(methoxypolyethylene glycol 5000 carbamoyl)-1,2-dipalmitoyl-sn-glycero-3-phosphatidylethanolamine (MPEG5000-DPPE), 1,2-dimyristoyl-sn-glycero-3-phosphoethanolamine-N-[methoxy(polyethylene glycol)-2000 (DMPE-PEG2000), 1,2-distearoyl-sn-glycero-3-phosphoethanolamine-N-[methoxy(polyethylene glycol)-2000 (DSPE-PEG2000), Polyoxyethylene 40 stearate (PEG40S) and combinations thereof.

23. The microbubble of claim 1 wherein the PEG or PEG-lipid is present in an amount of about 10 molar %.

24. The microbubble of claim 1, wherein the microbubble is substantially spherical and between about 0.8 μm-18 μm in diameter.

25. The microbubble of claim 1, wherein the microbubble is substantially spherical and between about 1 μm-9 μm in diameter.

26. The microbubble of claim 1, wherein the microbubble is substantially spherical and between about 2 μm-4 μm in diameter.

27. The microbubble of claim 1, wherein the porphyrin-phospholipid conjugate comprises a metal chelated therein, optionally a radioisotope of a metal.

28. The microbubble of claim 27 wherein the metal is selected from the group consisting of Zn, Cu, Mn, Fe and Pd.

29. The microbubble of claim 1, wherein the gas is a biologically inert gas.

30. The microbubble of claim 1, wherein the gas is a fluorinated gas.

31. The microbubble of claim 1, wherein the gas is selected from the group consisting of air, O2, N2, H2, CO2, N2O, noble gases, hydrocarbon gases, perfluorocarbon, sulphur hexafluoride, perfluoromethane, perfluoroethane, perfluoropropane, perfluorobutane and combinations thereof.

32. The microbubble of claim 1, wherein the remainder of the monolayer is comprised substantially of other phospholipid, preferably an anionic phospholipid.

33. The microbubble of claim 32, wherein the phospholipid is selected from the group consisting of selected from the group consisting of phosphatidylcholines, phosphatidylethanolamines, phosphatidic acid, phosphatidylglycerols and combinations thereof.

34. The microbubble of claim 32, wherein the phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-diligmoceroyl-sn-glycero-3-phosphatidylcholine(DLgPC), 1,2-dipalmitoyl-sn-glycero-3-[phosphor-rac-(1-glycerol)] (DPPG) and combinations thereof.

35. The microbubble of claim 1, wherein the microbubble is formed in solution comprising at least one additive selected from the group consisting of Sodium Chloride, Sodium Phosphate, Propylene glycol, Glycerol, Polyethylene glycol (preferably ranging from 1000 to 6000 molecular weight) and combinations thereof.

36. The microbubble of claim 1, further comprising a targeting molecule.

37. A method of preparing microbubbles, comprising mixing a gas, a porphyrin-phospholipid conjugate and another phospholipid, wherein the porphyrin-phospholipid conjugate comprises one porphyrin, porphyrin derivative or porphyrin analog covalently attached to a lipid side chain, preferably at the sn-1 or the sn-2 position, of one phospholipid.

38. The method of claim 37, wherein prior to mixing with the gas, the porphyrin-phospholipid conjugate is mixed with the other phospholipid to form a lipid film and then hydrated in buffer.

39. A method of performing ultrasound imaging on a target area in a subject comprising:
a. providing the microbubble of claim 1;
b. administering the microbubble to the subject; and
c. imaging the target area using ultrasound.

40. A method of imaging a target area in a subject, comprising
a. providing the microbubble of claim 1;
b. administering the microbubble to the subject; and
c. measuring and/or detecting a photoacoustic signal at the target area.

41. The method of claim 40, further comprising bursting the microbubble at the target area before step c.

42.-44. (canceled)
